(19) United States
(12) Patent Application Publication
(10) Pub. No.: US 2017/0354538 A1
(43) Pub. Date: Dec. 14, 2017

(54) EYEWEAR COMPONENT WITH REPLACEABLE LENS

(71) Applicant: ARTHUR WANG, TAIPEI CITY (TW)

(72) Inventor: ARTHUR WANG, TAIPEI CITY (TW)

(21) Appl. No.: 15/180,305

(22) Filed: Jun. 13, 2016

(51) Int. Cl. A61F 9/02

(52) U.S. Cl. CPC A61F 9/025

(57) ABSTRACT

Provided is an eyewear component with a replaceable lens, comprising a lens, a first buckle and a second buckle. The lens has a first end and a second end, the first end has an opening, and the second end has an elongated hole and a notch; the first buckle has a fixed element detachably received in the opening of the first end, and the fixed element has a head portion and a neck portion; the second buckle has an upper buckled portion and a lower buckled portion, the upper buckled portion and the lower buckled portion are detachably received in the elongated hole and the notch respectively. Therefore, the lens is not only easy to be buckled to the first buckle and the second buckle, but also easy to be detached for changing the lens.

### BACKGROUND OF THE INVENTION

### 1. Field of the Invention

The present invention relates to an eyewear component with a replaceable lens, particularly to a sunglasses component with a replaceable lens.

### 2. Description of the Prior Arts

Sport sunglasses are one type of the sunglasses for protecting eyes from excess ultraviolet (UV) light during outdoor activities. In different sports, the requirements of the sunglasses are not the same. For example, for activities in the snow or at a beach, the color depth and the UV filtration strength of the lens are both higher because the sunlight and the UV light are easy to reflect in the snow and at the beach. However, the eyes do not receive enough light when wearing the sport sunglasses for other occasions. Therefore, the sport sunglasses can only be used for a particular purpose.

As for the person who needs to participate in long time sports activities, such as a cyclist, the intensities of the sunlight are different at different times and through different terrains, such that the sunglasses of a mono-color depth cannot offer the most suitable protection throughout the activity. However, carrying around multiple sunglasses seems burdensome and unnecessary.

Therefore, the disadvantages in prior arts should be resolved.

### SUMMARY OF THE INVENTION

According to the above description, the objective of the present invention is to provide an eyewear component with a replaceable lens, which is easy to assemble and detach for the user to easily change the lens.

To achieve the above objective of the present invention, the invention provides an eyewear component with a replaceable lens, comprising a lens, a first buckle and a second buckle. The lens has a first end and a second end, the first end has an opening formed in the first end, and the second end has an elongated hole and a notch formed in the second end; the first buckle has a fixed element detachably received in the opening of the first end, and the fixed element has a head portion and a neck portion; the second buckle has an upper buckled portion and a lower buckled portion, and the upper buckled portion and the lower buckled portion are detachably received in the elongated hole and the notch of the second end respectively.

Therefore, the advantages of the present invention are that the lens, the first buckle, and the second buckle are easy to be assembled by the combination of the fixed element of the first buckle and the first end of the lens, and by the combination of the upper buckled portion and the lower buckled portion of the second buckle with the second end of the lens. Therefore, the lens is not only easy to be buckled to the first buckle and the second buckle, but also easy to be detached for changing the lens by the user.

Preferably, a width of the neck portion between an upper side and a lower side of the neck portion is wider than a width of the neck portion between a right side and a left side of the neck portion.

More preferably, a tapered portion and a receiving portion are formed at the opening of the lens, a width of the receiving portion between an upper side and a lower side of the receiving portion is equal to the width of the neck portion between the upper side and the lower side of the neck portion, and a smallest width of the tapered portion is equal to the width of the neck portion between the right side and the left side of the neck portion.

Preferably, the elongated hole of the second end of the lens and the notch are misaligned.

Preferably, a width of a lower end of the elongated hole near the notch is wider than a width of an upper end of the elongated hole opposite the lower end.

Preferably, the upper buckled portion of the second buckle has an annular flange formed at an end of the upper buckled portion.

Preferably, the lower buckled portion of the second buckle has a protrusion formed at an end and at a side of the lower buckled portion.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an eyewear component with a replaceable lens of the present invention;

FIG. 2 is another perspective view of the eyewear component with a replaceable lens of the present invention;

FIG. 3 is an exploded and perspective view of the eyewear component with a replaceable lens of the present invention;

FIG. 4A and FIG. 4B are cross-sectional views of the fixed element of the first buckle of the eyewear component with a replaceable lens of the present invention;

FIG. 5 is another exploded and perspective view of the eyewear component with a replaceable lens of the present invention;

FIG. 6 is a front view of the upper buckled portion and the lower buckled portion of the second buckle of the eyewear component with a replaceable lens of the present invention;

FIG. 7 is an enlarged and cross-sectional view taken along line A-A of FIG. 6 of the upper buckled portion and the lower buckled portion of the second buckle of the eyewear component with a replaceable lens of the present invention;

FIG. 8 is an enlarged and cross-sectional view taken along line B-B of FIG. 6 of the lower buckled portion of the second buckle of the eyewear component with a replaceable lens of the present invention.

### DETAILED DESCRIPTION OF THE PREFERRED EMBODIMENTS

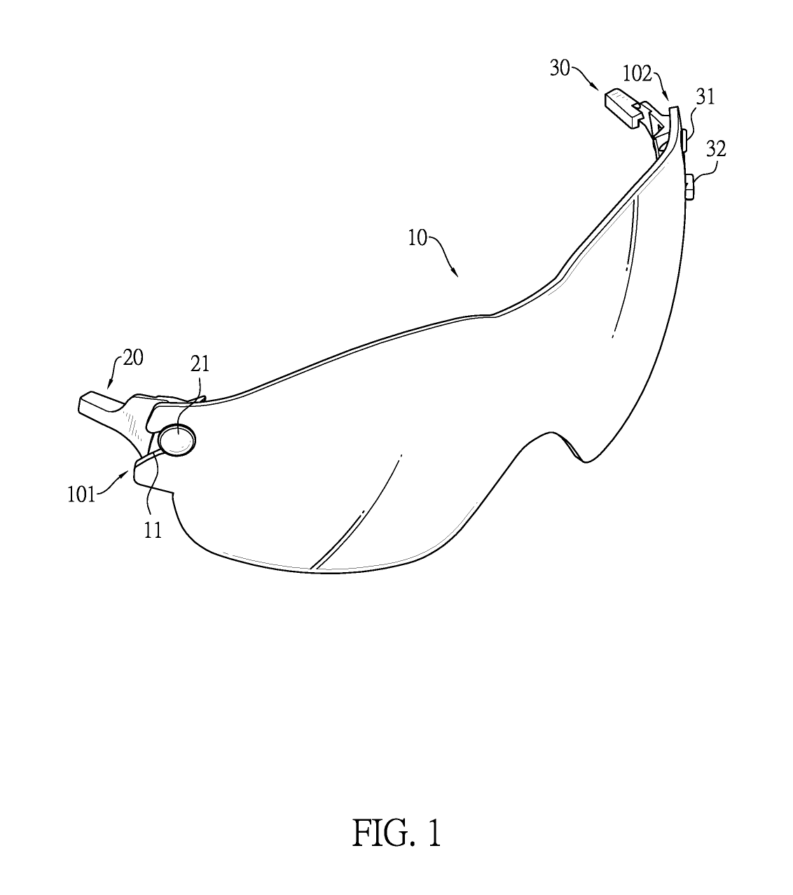

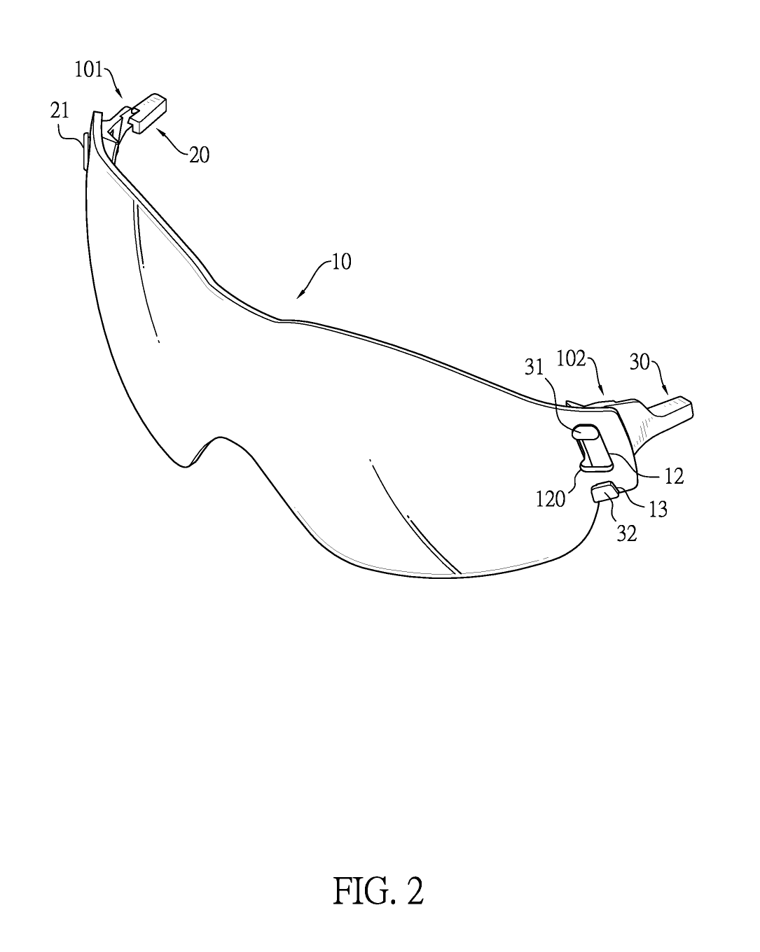

As shown in FIG. 1 and FIG. 2, the eyewear component with a replaceable lens of the present invention comprises a lens 10, a first buckle 20, and a second buckle 30.

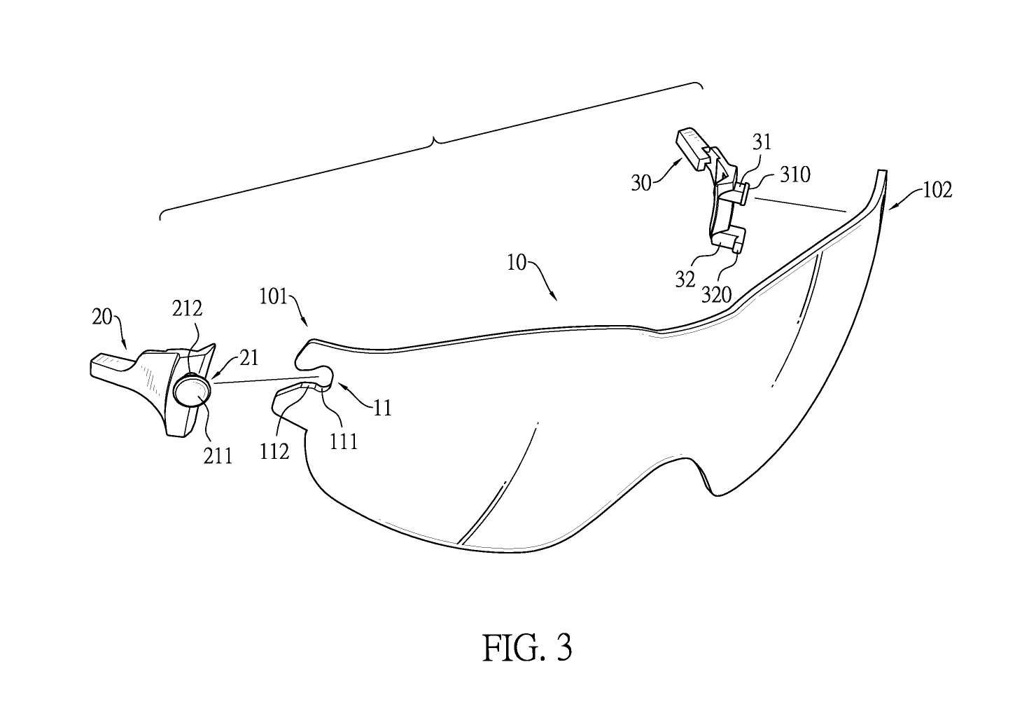

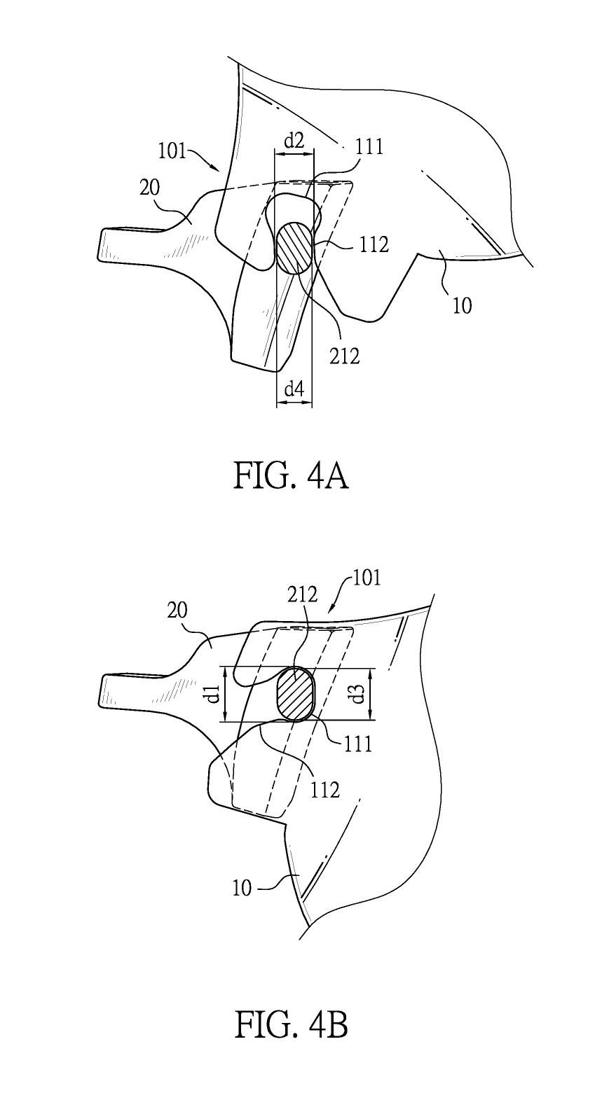

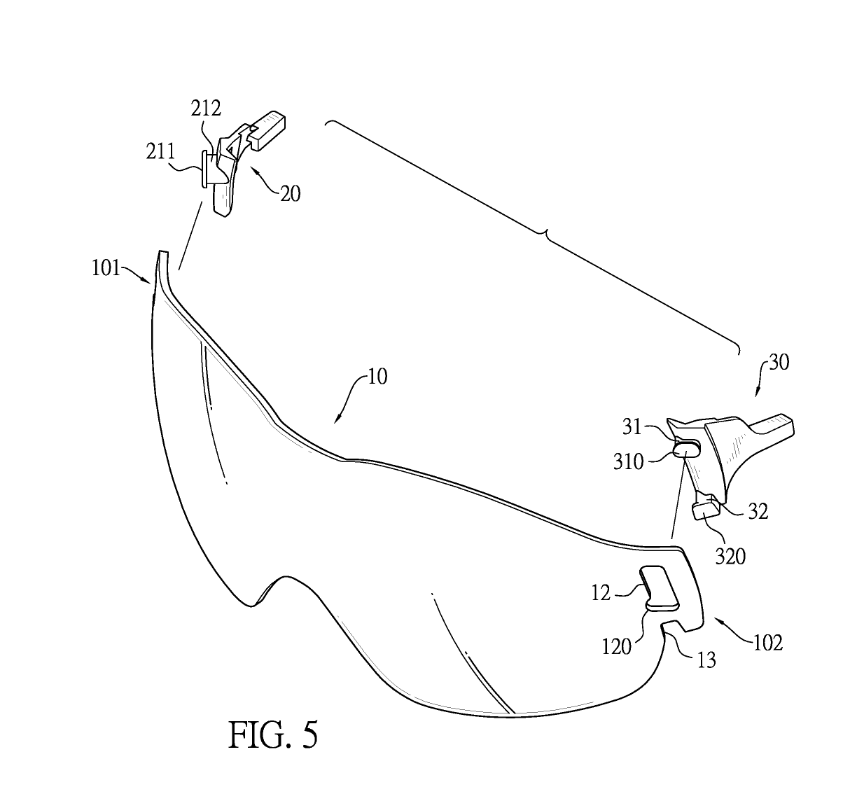

Two ends of the lens 10 are respectively defined as a first end 101 and a second end 102, the first end 101 is detachably located at the first buckle 20, and the second end 102 is detachably located at the second buckle 30. The first buckle 20 and the second buckle 30 can respectively be connected to the temples (not shown in the figures) to form a complete eyewear. As shown in FIG. 3, the first end 101 has an opening 11 formed in the first end 101 and extending along an axial direction of lens 10, and the opening 11 forms a receiving portion 111 and a tapered portion 112. As shown in FIG. 4A, and FIG. 4B, the receiving portion 111 is located at the widest portion of the opening 11 and has a first width d1 between an upper side and a lower side of the receiving portion 111, and the tapered portion 112 has a second width d2, which is the smallest width of the tapered portion 112. The first width d1 is wider than the second width d2. As shown in FIG. 5, the second end 102 of the lens 10 has an elongated hole 12 and a notch 13 formed in the second end 102. The elongated hole 12 has an upper end and a lower end, and extends from the upper end toward the lower end. A width of the lower end of the elongated hole 12 is wider than a width of the upper end of the elongated hole 12. Specifically, the lower end of the elongated hole 12 laterally forms a widened portion 120 such that the width of the lower end of the elongated hole 12 is wider than the width of the upper end of the elongated hole 12. The notch 13 is located at a lower end of the lens 10; in a preferred embodiment, the elongated hole 12 and the notch 13 are misaligned. Specifically, a center of the notch 13 is slightly away from the extending direction of the elongated hole 12; in another preferred embodiment, the notch 13 is on the extending direction of the elongated hole 12.

As shown in FIG. 4A, FIG. 4B, and FIG. 5, the first buckle 20 has a fixed element 21 detachably received in the receiving portion 111 of the opening 11 of the first end 101, and the fixed element 21 has a head portion 211 and a neck portion 212. In the preferred embodiment, the neck portion 212 has a third width d3 between an upper side and a lower side of the neck portion 212 (as shown in FIG. 4B) and a fourth width d4 between a right side and a left side of the neck portion 212 (as shown in FIG. 4A), and the third width d3 is wider than the fourth width d4. The third width d3 of the neck portion 212 and the first width d1 of the receiving portion 111 of the opening 11 are equal, and the fourth width d4 of the neck portion 212 and the second width d2 of the tapered portion 112 of the opening 11 are equal. The distance between the head portion 211 and the first buckle 20 is equal to the thickness of the lens 10, such that the lens 10 can be fastened by the head portion 211 and the first buckle 20.

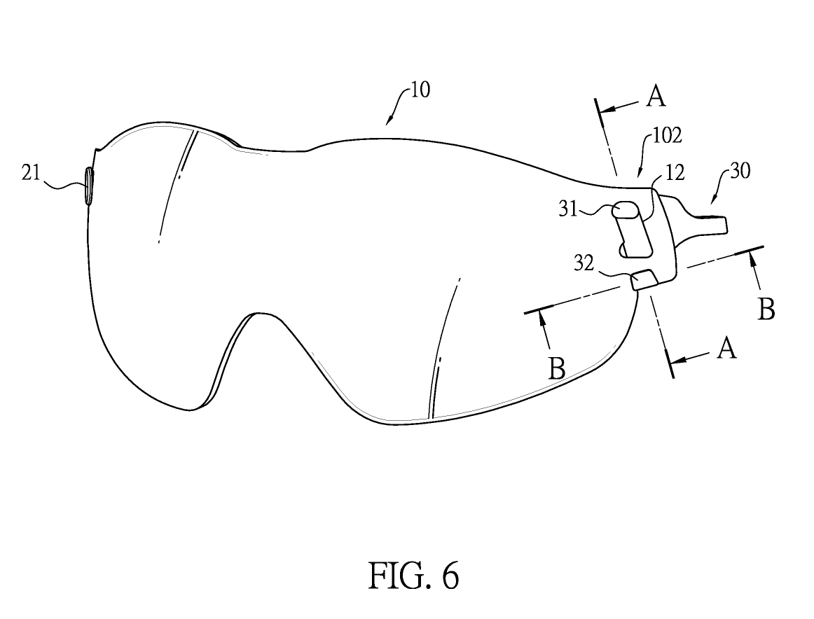

As shown in FIG. 5 and FIG. 6, the second buckle 30 has an upper buckled portion 31 and a lower buckled portion 32, the upper buckled portion 31 and the lower buckled portion 32 are detachably received in the elongated hole 12 and the notch 13 of the second end 102 respectively. The upper buckled portion 31 and the lower buckled portion 32 are respectively buckled on the elongated hole 12 and the notch 13 of the second end 102. Due to the misalignment of the elongated hole 12 and the notch 13, the lens 10 and the second buckle 30 are difficult to separate by a force applied from one single direction. The larger the misalignment, the more difficult the separation of the lens 10 and the second buckle 30.

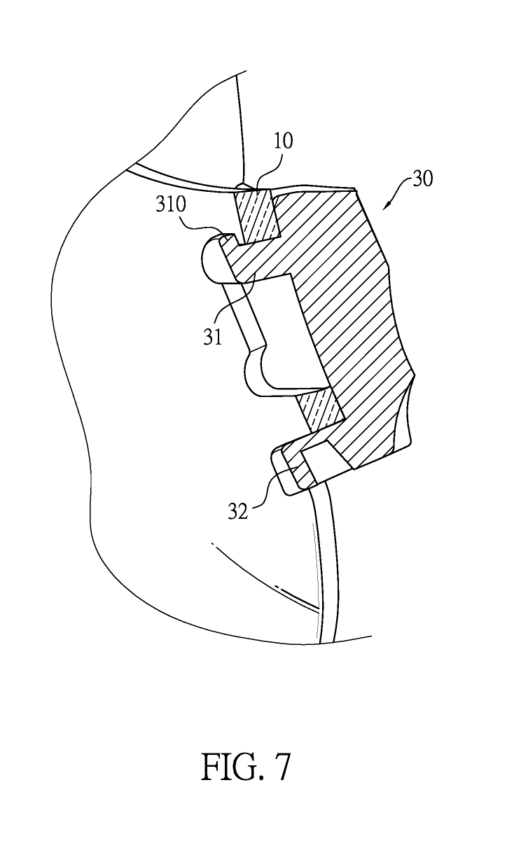

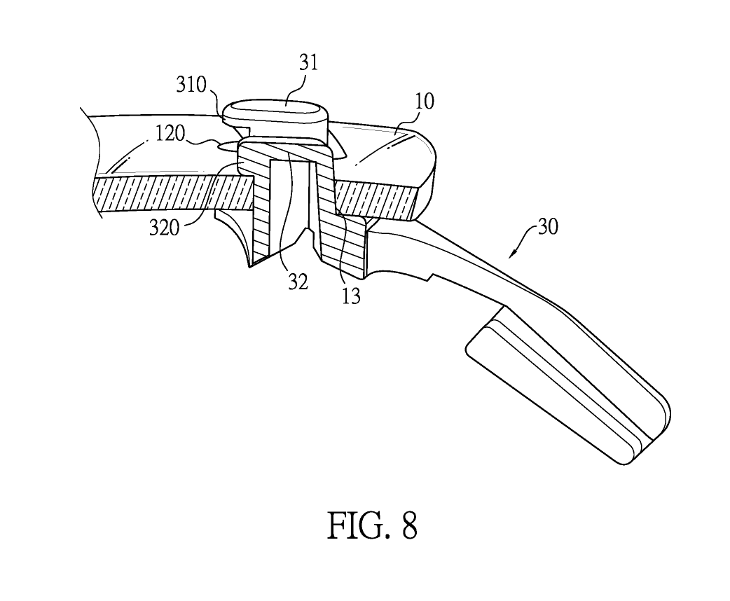

As shown in FIG. 7 and FIG. 8, the upper buckled portion 31 and the lower buckled portion 32 protruding away from the second buckle 30 are respectively inserted and received in the elongated hole 12 and the notch 13. As shown in FIG. 6 and FIG. 7, the upper buckled portion 31 has an annular flange 310 formed at an end of the upper buckled portion 31. In another preferred embodiment, the annular flange 310 is located at the end and at a side of the upper buckled portion 31, and specifically, the side of the upper buckled portion 31 is away from a side of the second end 102 of the lens 10. A widest width of the annular flange 310 and the width of the lower end of the elongated hole 12 are equal, and the widest width of the annular flange 310 is wider than the width of the upper end of the elongated hole 12 such that the annular flange 310 may be inserted through the lower end of the elongated hole 12 rather than through the upper end of the elongated hole 12. A width of the upper buckled portion 31 without the annular flange 310 and the width of the upper end of the elongated hole 12 are equal, and the distance between the annular flange 310 and the second buckle 30 is equal to the thickness of the lens 10. Therefore, the upper buckled portion 31 may be received at the upper end of the elongated hole 12, and the annular flange 310 and the second buckle 30 can be buckled on the lens 10.

As shown in FIG. 8, the lower buckled portion 32 of the second buckle 30 has a protrusion 320 formed at an end and at a side of the lower buckled portion 32. In other words, the protrusion 320 laterally protrudes from the lower buckled portion 32. Similarly, the protrusion 320 protrudes away from the side of the second end 102 of the lens 10, and the distance between the protrusion 320 and the second buckle 30 is equal to the thickness of the lens 10. Therefore, the protrusion 320 and the second buckle 30 can be buckled on the lens 10.

As shown in FIG. 3, when the user would like to combine the lens 10 with the first buckle 20 and the second buckle 30, the lens 10 is rotated to make the opening 11 disposed downwards, and the neck portion 212 of the fixed element 21 of the first buckle 20 is inserted through the tapered portion 112 of the opening 11 (as shown in FIG. 4A) for the neck portion 212 to be received in the receiving portion 111 of the opening 11. Next, the lens 10 is rotated to make the opening 11 disposed horizontally as shown in FIG. 3 and FIG. 4B. The fixed element 21 is difficult to detach because the smallest width of the tapered portion 112 (the second width d2) is shorter than the upper side and the lower side of the neck portion 212 (the third width d3). Besides, the first buckle 20 and the lens 10 are stably fixed by the head portion 211 and the first buckle 20 to be buckled on the lens 10 because the distance between the head portion 211 and the first buckle 20 are equal to the thickness of the lens 10.

As shown in FIG. 6 to FIG. 8, the upper buckled portion 31 of the second buckle 30 is inserted through the lower end of the elongated hole 12 of the lens 10, and the lens 10 is relative to the second buckle 30 and downwardly moved for the upper buckled portion 31 to receive the upper end of the elongated hole 12. In a preferred embodiment, the protrusion 320 of the lower buckled portion 32 abuts an edge of the notch 13 rather than being received inside the notch 13 due to misalignment between the elongated hole 12 and the notch 13. Therefore, by pushing the lens 10, the edge of the notch 13 is forced across the protrusion 320, such that the lower buckled portion 32 is received into the notch 13 (as shown in FIG. 8). The annular flange 310, the protrusion 320, and the second buckle 30 are buckled on the lens 10 because the distances between the annular flange 310 and the second buckle 30 and the distances between the protrusion 320 and the second buckle 30 are equal to the thickness of the lens 10.

Therefore, the complete eyewear is composed of the lens 10, the first buckle 20, the second buckle 30, and two temples (not shown in figures). The lens 10 can be detached by reversing the above-described steps.

In summary, the lens 10, the first buckle 20, and the second buckle 30 of the present invention are easy to be assembled by the combination of the fixed element 21 of the first buckle 20 and the first end 101 of the lens 10, and by the combination of the upper buckled portion 31 and the lower buckled portion 32 of the second buckle 30 with the second end 102 of the lens 10. Therefore, the lens 10 is not only easy to be buckled to the first buckle 20 and the second buckle 30, but also easy to be detached for changing the lens 10 by the user.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An eyewear component with a replaceable lens, comprising:
a lens having a first end and a second end, the first end having an opening formed in the first end, and the second end having an elongated hole and a notch formed in the second end;
a first buckle having a fixed element detachably received in the opening of the first end, and the fixed element having a head portion and a neck portion; and
a second buckle having an upper buckled portion and a lower buckled portion, and the upper buckled portion and the lower buckled portion detachably received in the elongated hole and the notch of the second end respectively.

2. The eyewear component with a replaceable lens as claimed in claim 1, wherein a width of the neck portion between an upper side and a lower side of the neck portion is wider than a width of the neck portion between a right side and a left side of the neck portion.

3. The eyewear component with a replaceable lens as claimed in claim 2, wherein a tapered portion and a receiving portion are formed at the opening of the lens, a width of the receiving portion between an upper side and a lower side of the receiving portion is equal to the width of the neck portion between the upper side and the lower side of the neck portion, and a smallest width of the tapered portion is equal to the width of the neck portion between the right side and the left side of the neck portion.

4. The eyewear component with a replaceable lens as claimed in claim 1, wherein the elongated hole of the second end of the lens and the notch are misaligned.

5. The eyewear component with a replaceable lens as claimed in claim 2, wherein the elongated hole of the second end of the lens and the notch are misaligned.

6. The eyewear component with a replaceable lens as claimed in claim 3, wherein the elongated hole of the second end of the lens and the notch are misaligned.

7. The eyewear component with a replaceable lens as claimed in claim 1, wherein a width of a lower end of the elongated hole near the notch is wider than a width of an upper end of the elongated hole opposite the lower end.

8. The eyewear component with a replaceable lens as claimed in claim 2, wherein a width of a lower end of the elongated hole near the notch is wider than a width of an upper end of the elongated hole opposite the lower end.

9. The eyewear component with a replaceable lens as claimed in claim 3, wherein a width of a lower end of the elongated hole near the notch is wider than a width of an upper end of the elongated hole opposite the lower end.

10. The eyewear component with a replaceable lens as claimed in claim 4, wherein a width of a lower end of the elongated hole near the notch is wider than a width of an upper end of the elongated hole opposite the lower end.

11. The eyewear component with a replaceable lens as claimed in claim 5, wherein a width of a lower end of the elongated hole near the notch is wider than a width of an upper end of the elongated hole opposite the lower end.

12. The eyewear component with a replaceable lens as claimed in claim 6, wherein a width of a lower end of the elongated hole near the notch is wider than a width of an upper end of the elongated hole opposite the lower end.

13. The eyewear component with a replaceable lens as claimed in claim 1, wherein the upper buckled portion of the second buckle has an annular flange formed at an end of the upper buckled portion.

14. The eyewear component with a replaceable lens as claimed in claim 2, wherein the upper buckled portion of the second buckle has an annular flange formed at an end of the upper buckled portion.

15. The eyewear component with a replaceable lens as claimed in claim 3, wherein the upper buckled portion of the second buckle has an annular flange formed at an end of the upper buckled portion.

16. The eyewear component with a replaceable lens as claimed in claim 10, wherein the upper buckled portion of the second buckle has an annular flange formed at an end of the upper buckled portion.

17. The eyewear component with a replaceable lens as claimed in claim 1, wherein the lower buckled portion of the second buckle has a protrusion formed at an end and at a side of the lower buckled portion.

18. The eyewear component with a replaceable lens as claimed in claim 2, wherein the lower buckled portion of the second buckle has a protrusion formed at an end and at a side of the lower buckled portion.

19. The eyewear component with a replaceable lens as claimed in claim 3, wherein the lower buckled portion of the second buckle has a protrusion formed at an end and at a side of the lower buckled portion.

20. The eyewear component with a replaceable lens as claimed in claim 16, wherein the lower buckled portion of the second buckle has a protrusion formed at an end and at a side of the lower buckled portion.
